SEQUENCE LISTING (12 sequences)

SEQ ID NO: 1 - DNA, 1080 bp, Artificial sequence
  Description of the artificial sequence Coding DNA sequence and/or translated amino acid sequence of the fusion protein CD40ex-Flag-FasL (construct A) according to the invention.
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgggatccgattacaaagacgatgacgataaagaattcggccggggcgactcacccggccggggcgactcacccgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctctaa

SEQ ID NO: 2 - protein, 359 aa, Artificial sequence
  Description of the artificial sequence Coding DNA sequence and/or translated amino acid sequence of the fusion protein CD40ex-Flag-FasL (construct A) according to the invention.
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLGSDYKDDDDKEFGRGDSPGRGDSPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 3 - DNA, 1176 bp, Artificial sequence
  Description of the artificial sequence Coding sequence and/or translated amino acid sequence of the fusion protein CD40ex-Flag-TRAIL (construct B) according to the invention.
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgggatccgattacaaagacgatgacgataaagaattcacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 4 - protein, 391 aa, Artificial sequence
  Description of the artificial sequence Coding sequence and/or translated amino acid sequence of the fusion protein CD40ex-Flag-TRAIL (construct B) according to the invention.
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLGSDYKDDDDKEFTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 5 - protein, 24 aa, Artificial sequence
  Description of the artificial sequence Linker1 in construct A (this peptide sequence may conceivably occur naturally).
GSDYKDDDDKEFGRGDSPGRGDSP

SEQ ID NO: 6 - protein, 12 aa, Artificial sequence
  Description of the artificial sequence Linker1 in construct B (this peptide sequence may conceivably occur naturally).
GSDYKDDDDKEF

SEQ ID NO: 7 - DNA, 72 bp, Artificial sequence
  Description of the artificial sequence Primer 1106
ccggaattcggccggggcgactcacccggccggggcgactcacccgaaaaaaaggagctgaggaaagtggcc

SEQ ID NO: 8 - DNA, 37 bp, Artificial sequence
  Description of the artificial sequence Primer 1107
ccgctcgaggtgcttctcttagagcttatataagccg

SEQ ID NO: 9 - DNA, 36 bp, Artificial sequence
  Description of the artificial sequence Primer 1160
cccaagcttctcgccatggttcgtctgcctctgcag

SEQ ID NO: 10 - DNA, 33 bp, Artificial sequence
  Description of the artificial sequence Primer 1161
cgcggatcccagccgatcctggggaccacagac

SEQ ID NO: 11 - DNA, 46 bp, Artificial sequence
  Description of the artificial sequence Primer 1302
ccggaattctacgcatattacacctctgaggaaaccatttctacag

SEQ ID NO: 12 - DNA, 36 bp, Artificial sequence
  Description of the artificial sequence Primer 1334
tgctctagaccaggtcagttagccaactaaaaaggc

(19) United States
(12) Patent Application Publication
(10) Pub. No.: US 2005/0244370 A1
(43) Pub. Date: Nov. 3, 2005

(54) Selective local activation of members of the TNF receptor family by systemically inactive non-antibody TNF ligand fusion proteins

(76) Inventors: Klaus Pfizenmaier, Tiefenbronn (DE); Harald Wajant, Leinfelden-Echterdingen (DE)

Correspondence Address: KLAUBER & JACKSON, 411 HACKENSACK AVENUE, HACKENSACK, NJ, 07601, US

(21) Appl. No.: 11/105,172

(22) Filed: Apr. 13, 2005

Related U.S. Application Data
(63) Continuation of application No. PCT/EP03/11357, filed on Oct. 14, 2003.

(30) Foreign Application Priority Data
Oct. 14, 2002 (DE) 10247755.8

(51) Int. Cl. A61K 38/19; A61K 38/18; C07H 21/04; C12P 21/06

(52) U.S. Cl. 424/85.1; 530/351; 530/399; 435/69.1; 435/320.1; 435/325; 536/23.5; 514/12

(57) ABSTRACT

Selective local activation of members of the TNF receptor family by systemically inactive non-antibody TNF ligand fusion proteins The present invention relates to polypeptides consisting of an effector binding domain and a cell surface molecule binding domain, which are coupled by a peptide linker. The effector domain is a fragment, especially the extracellular domain, of a member of the TNF ligand family (module 1), which as such is biologically inactive or of restricted activity. The cell surface molecule binding domain (module 2) is an amino acid segment that binds selectively to a surface structure of the plasma membrane, preferably to a membrane protein, but is not derived from an immuno-globulin. The invention also provides nucleic acid constructs coding for the polypeptides, vectors containing said constructs, host cells transfected with said vectors, pharmaceutical compositions containing said subjects of the invention, processes for the preparation of the polypeptides according to the invention, and uses of subjects of the invention for therapeutic purposes.

The present application is a Continuation of co-pending PCT Application No. PCT/EP2003/011357, filed Oct. 14, 2003 which in turn, claims priority from German Application Serial No. 10247755.8, filed Oct. 14, 2002. Applicants claim the benefits of 35 U.S.C. §120 as to the PCT application and priority under 35 U.S.C. §119 as to said German application, and the entire disclosures of both applications are incorporated herein by reference in their entireties.

The present invention relates to polypeptides consisting of an effector binding domain and a cell surface molecule binding domain, which are coupled by a peptide linker. The effector domain is a fragment, especially the extracellular domain, of a member of the TNF ligand family (module 1), which as such is biologically inactive or of restricted activity. The cell surface molecule binding domain (module 2) is an amino acid segment that binds selectively to a surface structure of the plasma membrane, preferably to a membrane protein, but is not derived from an immunoglobulin. The invention also provides nucleic acid constructs coding for the polypeptides, vectors containing said constructs, host cells transfected with said vectors, pharmaceutical compositions containing said subjects of the invention, processes for the preparation of the polypeptides according to the invention, and uses of subjects of the invention for therapeutic purposes.

In in vitro experiments, cytokines such as members of the TNF ligand family, for example TRAIL (TNF Related Apoptosis Inducing Ligand), also called Apo2L (Wiley et al. (1995) Immunity 6, 673-682; Pitti et al. (1996) J. Biol. Chem. 271, 12687-12689), and FasL, exhibit a potent apoptotic action on many tumour cells of animal and human origin. In the case of TRAIL, it appears that non-malignant cells are not damaged. Moreover, in the preclinical animal models studied (mouse, monkey), no indications whatsoever were found for an acute toxicity or other systemic side effects of TRAIL which could be regarded as restricting therapy (Walczak et al. (1999) Nat. Med. 5, 157-163; Ashkenazi et al. (1999) J. Clin. Invest. 104, 155-162). However, more recent in vitro experiments on primary human hepatocytes showed a potent cytotoxic action of e.g. a TRAIL product prepared by recombination or membrane-based TRAIL of the naturally occurring form of the cytokine TRAIL. (Jo et al. (2000) Nat. Med. 6, 564-567; Ichikawa et al. (2001) Nat. Med. 7, 954-960). Thus, a clinical systemic use of special soluble variants of members of the TNF ligand family (especially TNF, FasL, TRAIL, CD40L), which are bioactive and therefore have substantially the same action as the membrane-based ligand, is currently ruled out because of the side effects that occur. The same applies to agonistic antibodies that activate the receptors corresponding to these ligands. Thus, for example, FasL (ligand of the Fas receptor (Fas, CD95)), the prototype of apoptotic cytokines, was not used clinically, a priori on safety grounds, because agonistic antibodies to its receptor, Fas, are extremely hepatotoxic in vivo (Ogasawara et al. (1993) Nature 364, 806-809). Finally, it was also shown that FasL in soluble form possesses no bioactivity, in practical contrast to its membrane-based form (Schneider et al. (1998) J. Exp. Med. 187, 1205-1213).

Thus, the variants of the members of the TNF ligand family that are available according to the state of the art are unusable or only of very limited use (e.g. in the case of TNF under “isolated limb perfusion” conditions) for therapeutic application, for example for the treatment of tumours, either because of lack of bioactivity or because of extreme side effects.

WO 02/22680 discloses fusion proteins in which fragments of e.g. TNF cytokines that per se are biologically inactive or of restricted activity are bound via a peptide linker to an antigen-binding antibody or an antigen-binding antibody fragment. However, there is a need for alternative systems since it is not uncommon for there to be no appropriate antibodies (or antibody fragments) available for particular cell surface molecules or the antigen binding does not take place as expected, for example does not take place with the appropriate specificity.

Now, the object of the present invention is to provide an alternative system, especially one that is independent of antibodies, which makes it possible to develop the action of ligands of the TNF family (or fragments thereof) in a directed and tissue-specific or cell-specific manner and hence to avoid or at least greatly restrict unwanted and possibly systemic side effects on tissues/cells not belonging to the target tissue in a clinical application.

This object is achieved by the embodiments of the present invention characterized in the claims.

In particular, the invention provides a polypeptide comprising a segment (1), which contains a cell surface molecule binding domain (also called module 2), a segment (2), which is a peptide linker, and a segment (3), which contains a fragment of a member of the TNF ligand family (hereafter also called “TNF cytokines” or “TNF receptor ligand family”) or a functional variant thereof (also called module 1) that as such is biologically inactive/of restricted activity, the cell surface molecule binding domain not being derived from an immunoglobulin and the fragment of the member of the TNF ligand family or a functional variant thereof becoming biologically fully active only when segment (1) binds to the cell surface molecule.

In one preferred embodiment, segments (1) to (3) in the polypeptide (=fusion protein according to the invention=protein construct according to the invention) are arranged from N-terminal to C-terminal.

The present invention is based on the observation that, when they are proteolytically processed by the organism into a soluble form corresponding to the extracellular domain, ligands of the TNF family that occur naturally in membrane-based form are either biologically totally inactive or are only of restricted activity, e.g. on particular membrane receptor subtypes. This also applies to derivatives prepared by recombination that correspond to the extracellular domain of the ligand in question. Interestingly, such biologically inactive, soluble ligands are definitely still capable of binding to their complementary membrane receptor without however causing an activation of the receptor, i.e. signal transduction and initiation of a cellular reaction. It is known according to the inventive observation that, by fusion with another peptide component (module 2), which does not consist of an antibody or antibody fragment and which allows a specific binding, independent of the TNF ligand part, to a cell membrane-based structure, a ligand of the aforesaid type that is inactive/of restricted activity (module 1) again achieves (full) biological activity when this peptide component facilitates binding to a cell surface. The reconstitution of the biological action of module 1 which is achieved in this way becomes active both towards the target cells themselves, recognized by module 2, and towards adjacent cells, provided said cells express the appropriate receptors for the TNF ligand fragment (module 1) contained in the fusion protein.

The cell surface molecule binding domain (segment (1) or module (2)) of the fusion proteins according to the invention mediates the binding of the fusion protein to cells having the appropriate surface structure. By virtue of the specific modules, the invention is distinguished by two characteristics that are such as to give the whole protein properties which neither of the modules used possesses on its own and which cannot be anticipated.

The first of these characteristics is that the cell surface molecule binding domain in the fusion protein is not achieved by the principle corresponding to the state of the art whereby antibodies or fragments derived therefrom (e.g. scFv) are used, but that, rather than the conventionally used antigen-binding domains of antibodies, other cell surface molecule binding domains are unexpectedly capable of imparting a full and hence highly effective effector action, directed specifically against the target cells, to fragments of TNF cytokines that per se are biologically inactive/of restricted activity by binding to particular cell surface structures, especially membrane proteins. Examples of preferred non-immunoglobulin domains for binding to cell surface structures are peptide hormones or cytokines, short peptides and also receptors for membrane-based ligands (or, preferably, fragments thereof which undergo a specific binding).

The second characteristic consists in the fact that the effector domain, as such as well as in the fusion protein, is biologically inactive or of low activity on cells that do not possess the surface structure recognized by the cell surface molecule binding domain, i.e. the receptor corresponding to the effector domain is not activated. The surprising characteristic, inherent only to the whole fusion protein, is that the effector domain (fragment of a TNF cytokine that per se is biologically inactive/of restricted activity) of the fusion protein only becomes biologically active after binding to cells which express the surface structure recognized by the cell surface molecule binding domain. Thus the effector domain of the polypeptide according to the invention only activates the receptor corresponding to said domain on a cell which has the surface structure corresponding to the cell surface molecule binding module, or on a cell which is adjacent to a cell equipped with the surface structure. The fusion protein, but not its individual components (module 1 or module 2) nor an active form of the effector domain, thus selectively activates the receptor corresponding to the effector domain on cells (or cells adjacent thereto) which express the surface structure recognized by the cell surface molecule binding domain. This novel characteristic (selective local receptor activation) of the polypeptide according to the invention therefore makes it possible to avoid the systemic side effects exhibited by the active TNF cytokine from which the effector domain according to the invention was derived, or by agonistic antibodies known in the state of the art which activate the corresponding receptor. The polypeptide according to the invention thus allows the development of a targeted local action in the whole organism.

The cell surface molecule binding domain is advantageously chosen in such a way that the surface structure recognized by said domain is selectively present or at least concentrated on the cells, sites or organs on which the effector domain is to be active. The cell surface molecule binding domain is also chosen in such a way as not to allow autoactivation of the fusion protein, e.g. by aggregation.

Members of the TNF ligand family, for example TNF (Tumour Necrosis Factor; GenBank accession no. NM_{—}000594), TRAIL (TNF Related Apoptosis Inducing Ligand; GenBank accession no. U37518), also called Apo2L, CD40L (GenBank accession no. NM_{—}011616) and FasL (GenBank accession no. U11821), have an apoptotic and/or immune system-regulating action. Apart from a few exceptions, the members of the TNF ligand family are type II membrane proteins. In many cases, however, soluble forms can also be derived from these membrane-based forms by specific proteolysis or alternative splicing. In particular, soluble forms can also be prepared by genetic engineering methods. Soluble as well as membrane-based members of the TNF ligand family develop their biological action by interaction with the members of a corresponding family of receptors, namely the members of the TNF receptor family. In some cases the soluble and membrane-based forms of members of the TNF ligand family can differ considerably in their bioactivity. Thus, for example, soluble TNF is a very potent activator of TNF-R1 but has no action or only a very restricted action at TNF-R2. On the other hand, the membrane-based form of the same ligand can activate both TNF receptors equally well. Other examples where soluble and membrane-based forms of members of the TNF receptor ligand family differ are, inter alia, FasL, CD40L and TRAIL.

In one preferred embodiment, polypeptides according to the invention therefore contain, in their segment (3), an amino acid sequence of a soluble form of a member of the TNF receptor ligand family, a variant of such a sequence or a fragment thereof.

A variant is generally understood according to the invention as meaning sequences which contain at least parts, preferably at least 50% and particularly preferably at least 80% of the native sequence and differ from the native sequence by e.g. one or more deletions, one or more insertions and/or at least one mutation. The sequence homology with the appropriate native sequence is preferably at least 90%, particularly preferably at least 95% and very particularly preferably at least 97%. A functional fragment can be N-terminally, C-terminally or intra-sequentially shortened sequences of the soluble forms of the members of the TNF receptor ligand family. Biologically active variants of these fragments are also disclosed according to the invention. In terms of the invention, the derived variants will preferably have selective receptor binding properties, it being possible for the variant to be optimized e.g. in respect of its specific bioactivity or other properties (stability).

The mode of action of constructs according to the invention is especially applicable to all those members of the TNF receptor ligand family which are exclusively active or act particularly well as a membrane molecule for certain receptors. Apart from FasL and CD40L, these also include TNF, TRAIL, 41BB, CD30L and Ox40L. Particularly preferred members of the TNF receptor ligand family in module 1 (segment (3)) of the polypeptides according to the invention are therefore those which naturally activate their complementary receptor only as a membrane protein.

A very particularly preferred segment (3) is a soluble extracellular domain, a functional variant of a soluble extracellular domain or a functional fragment of a soluble extracellular domain of TRAIL, FasL, CD40, 41BBL, CD30L, Ox40L or TNF.

The linker segment (2) between segment (1) (cell surface molecule binding domain; module 2) and segment (3) (TNF receptor ligand fragment; module 1) takes the form of e.g. a flexible compound in polypeptide constructs according to the invention, but preferably without adversely influencing the intrinsic trimerization properties of the relevant TNF receptor ligand fragment (module 1), as shown below in exemplary constructs (A) and (B).

Any naturally occurring or synthetically prepared peptide sequence is conceivable in segment (2). In principle, a linker can correspond to a native or varied (partial) sequence of any organisms, preferably from vertebrates, particularly mammals and especially man. Examples of other suitable linkers are any sequence segments of proteins which generate trimers by the formation of supersecondary structures. In any case, the sequences of native polypeptides or fragments of these native polypeptides which are used as linkers in segment (2) of a polypeptide according to the invention can also occur in the form of biologically active variants thereof, in terms of the present invention and according to the above definition.

The cell surface molecule binding domain (segment (1)) of the polypeptide of the present invention can include e.g. that segment of a receptor for a membrane-based ligand which is required for the specific interaction with the cell surface molecule. Preferred polypeptides within the framework of the present invention are those which have a soluble ligand-binding domain of a receptor, especially of a member of the TNF receptor family. In this case the receptor part must not be complementary to the TNF receptor ligand fragment present in segment (3).

In another preferred embodiment of the polypeptide, segment (1) comprises at least that segment of a ligand of a membrane-based receptor which is required for the specific interaction with the cell surface molecule. Soluble ligands or peptides that recognize cell-based receptors are therefore suitable according to the invention. TNF receptor ligand fragments different from segment (3) (module 1) are also suitable. A polypeptide according to the invention will be very particularly preferred when segment (1) is a soluble mammalian ligand/receptor fragment, especially of human origin, and interacts specifically with defined complementary target structures on mammalian and especially human cells and tissues.

Segment (1) of a polypeptide according to the invention will preferably have specificity for a cell surface protein that is selectively or dominantly expressed in the tumour tissue or on activated cells of the immune system (e.g. T cells, B cells, macrophages, dendritic cells). Examples of such selectively or predominantly expressed cell surface proteins which can be bound by segment (1) of a polypeptide according to the invention are e.g. VEGFR or the VEGFR/VEGF complex, any mutated and wild-type forms of the EGF receptor family or CD30 in the case of tumour tissues, e.g. CD40L or the IL2 receptor in the case of activated T cells, e.g. Baff-R or CD40 in the case of B cells, e.g. membrane TNF or the B7 ligand in the case of macrophages, and again the B7 ligand in the case of dendritic cells.

The following are therefore found to be preferable for segment (1) of a polypeptide according to the invention: protein or peptide hormones, e.g. growth factors such as EGF, and angiogenesis factors such as VEGF, as well as the soluble extracellular domains of receptors such as any members of the TNF receptor superfamily, especially TNF-R2, CD30 and CD40, and of other receptors such as CD28.

A preferred polypeptide according to the invention thus takes the form of a recombinant fusion protein that basically contains the following structural elements (monomers) in a defined order: segment (1), which is e.g. N-terminal and contains a ligand or receptor fragment or a peptide; segment (2), which contains a linker sequence; and segment (3), which is e.g. C-terminal and contains the human extracellular domain of FasL, TNF or CD40L. Analogously, it is possible to use e.g. CD30L, Ox40L or other members of the TNF family as segment (3) in appropriate polypeptides according to the invention.

The present invention also provides nucleic acids having nucleotide sequences (especially DNA sequences) that code for fusion proteins of the above-mentioned type according to the invention (nucleic acid constructs) or contain such a region coding for a polypeptide according to the invention. Such nucleic acid constructs are preferably expressed in expression vectors. Thus the present invention also provides the appropriate vectors containing a DNA sequence for the fusion proteins according to the invention. Preferably, vectors according to the invention are capable of expressing and/or amplifying in a prokaryotic and/or eukaryotic cell. In particular, the present invention further relates to retroviral vectors as well as any vector systems that can be used for gene therapy, including e.g. adenoviral vector systems. Thus, within the framework of the present invention, methods of gene therapy with vectors or nucleic acid constructs according to the invention are also disclosed as methods of treatment for the medical indications disclosed according to the invention.

The present invention also provides host cells that are transfected with nucleic acids coding for the fusion proteins according to the invention. Very particularly preferred host cells in this connection are those which are transfected with expression vectors according to the invention or nucleic acid constructs according to the invention, the expression vectors in turn containing DNA sequences coding for the fusion proteins according to the invention.

The present invention also provides processes for the preparation (expression and isolation) of polypeptides according to the invention, an isolation process according to the invention typically being characterized by (a) provision of a vector according to the invention or a nucleic acid construct, (b) transfection of cells with a vector or nucleic acid construct obtained according to process step (a), (c) cultivation of the cells transfected according to (b), and (d) isolation of polypeptides according to the invention, expressed under appropriate conditions, from the host cells and/or the culture supernatant. The expression of the fusion protein typically takes place according to the state of the art in suitable expression systems, preferably as a secreted product of stable transfectants, e.g. CHO cells, or in other animal cells, such as Cos7 or SF9 (insect cells), or other eukaryotic cell systems, especially in yeasts, e.g. Pichia pastoris. Preferably, the expressed polypeptides according to the invention will contain appropriate leader sequences suitable for secretion in the cell system. The vectors according to the invention used for expression will therefore also contain coding segments that code for a functional leader sequence.

Polypeptides according to the invention, and optionally also nucleic acid constructs, vectors or host cells (combined here in the category “substances according to the invention” or “subjects of the invention”), are also considered as drugs or for the preparation of a drug. They are used in particular in cases where, after binding of the fusion protein via its module 2 (segment (1)) to a specific (cell) membrane-based target molecule, substances according to the invention are to develop a biological action via the corresponding receptor of the TNF receptor ligand fragment according to module 1 (segment (3)). By choosing module 2 appropriately, the TNF receptor ligand activity of the substance according to the invention is directed at the tissue to be treated or the cell type to be treated, and it is possible to prepare a therapeutic agent that is specifically adapted/optimized to the appropriate indication.

When used e.g. as a therapeutic agent for tumours, especially for the treatment of solid tumours, but also lymphatic tumours (benign or malignant), a polypeptide according to the invention, after in vivo administration, is first specifically bound in the tumour area itself or to the reactive stroma/vascular system of the tumour via the cell surface molecule binding domain (module 2), and thereby activated. The receptors corresponding to the TNF receptor ligand fragment (module 1) can then be activated and induce e.g. apoptosis (module 1=e.g. soluble fragment of FasL or TRAIL) or activate infiltrating T cells (module 1=e.g. soluble fragment of CD40L). When used e.g. as a therapeutic agent in autoimmune diseases, a polypeptide according to the invention, after in vivo administration, is first bound to a surface marker of activated T cells (e.g. CD40L) or activated macrophages (e.g. membrane TNF) via the cell surface molecule binding domain (module 2), and thereby becomes biologically fully active. The receptors corresponding to the TNF receptor ligand fragment (module 1) can then be activated and induce e.g. apoptosis (module 1=e.g. soluble fragment of FasL or TRAIL).

In principle, however, it is also always desirable to use substances according to the invention in the field of therapeutics when the activation of a signal transduction chain such as the signal cascades triggered by the TNF receptor family, e.g. an apoptotic signal cascade, is to be initiated. Thus the use of substances according to the invention is considered in the treatment of any hyperproliferative diseases, or for the preparation of a drug for said treatment, including, for example, for the targeted elimination of cells of the immune system in the case of excessive immune reactions, for example in autoimmune diseases such as multiple sclerosis, rheumatoid arthritis, diabetes mellitus and TEN, or in the case of misdirected immune reactions against foreign antigens, such as those which can occur e.g. in infectious diseases (bacterial (caused, for example, by mycobacteria), viral or protozoological). Also considered is the treatment of metabolic diseases or general hyperinflammatory states, especially chronic inflammations, including, for example, in the case of allergies, as well as the treatment of rejection reactions by the patient's immune system against foreign tissue. In the above-mentioned cases, the cell surface molecule binding domain (module 2), i.e. segment (1), of a polypeptide according to the invention must always recognize characteristic markers on the surface of the target cells in which preferably an apoptotic signal cascade, aimed at cell death, is to be triggered. Thus, for example, in the case of treatment following the transplantation of foreign tissue, the endogenous cells of the transplant patient's immune system that are responsible for the rejection reaction are used as target cells. Likewise, for example, in the case of the treatment of rheumatoid arthritis, the endogenous activated macrophages that play a substantial part in the disease are used as target cells.

As explained above, subjects of the invention, such as nucleic acid constructs, expression vectors or host cells, are also considered as drugs e.g. for the treatment of the above-mentioned diseases. In this case, cells to be transfected are preferably taken from the patient to be treated, transfected in vitro with expression vectors according to the invention, cultivated and then introduced into the patient as an autotransplant (retransplant). The transfection is preferably performed by means of nucleic acid constructs or expression vectors according to the invention which couple the expression to a regulatable promoter. The transfected autotransplant can be administered locally, for example injected, depending on the specific disease and the specific target cells. A local administration is preferred e.g in the case of a tumour therapy. Here, tumour cells are taken from the patient, transfected in vitro and then preferably injected directly into the tumour. This procedure is suitable e.g. for the treatment of skin tumours (e.g. melanomas), tumours of the nervous system (e.g. glioblastomas), etc.

The present invention also provides a pharmaceutical composition containing polypeptides according to the invention, nucleic acid constructs according to the invention, vectors according to the invention and/or host cells according to the invention, together with pharmaceutically acceptable auxiliary substances, additives and/or excipients (including e.g. solubilizers). Thus a combination of substances according to the invention with pharmaceutically acceptable excipients, auxiliary substances and/or additives is disclosed according to the invention. Appropriate methods of preparation are disclosed in “Remington's Pharmaceutical Sciences” (Mack Pub. Co., Easton, Pa., 1980), which is part of the disclosure of the present invention. Examples of possible excipients for parenteral administration are sterile water, sterile saline solutions, polyalkylene glycols, hydrogenated naphthalene and especially biocompatible lactide polymers, lactide/glycolide copolymers or polyoxyethylene/polyoxypropylene copolymers. Such compositions according to the invention are considered for all the medical indications disclosed above.

In principle, within the framework of the present invention, all the modes of administration known in the state of the art are disclosed for substances according to the invention or compositions according to the invention. Preferably, a drug for the treatment of the above-mentioned diseases or disorders is administered by the parenteral route, for example subcutaneously, intramuscularly or intravenously, or by the oral or intranasal route. Typically, pharmaceutical compositions according to the invention will be solid, liquid or in the form of an aerosol (e.g. formulated as a spray). The form of the composition will vary according to the type of formulation.

In summary, it should be stated that, according to the invention, dual-specific fusion proteins with proapoptotic and immunomodulating properties are provided which contain soluble fragments of a priori membrane-based TNF receptor ligands that are inactive or of restricted activity, but which can be activated locally by binding to cell surface structures that do not interact with the TNF receptor ligand fragment. The cell surface molecule binding domain can itself be a second cytokine, a receptor for membrane-based ligands or another non-antibody peptide (or a fragment thereof) that undergoes a specific binding to cell membrane structures. Via this cell surface molecule binding domain, the TNF receptor ligand activity is directed to the tissue to be treated or the cell types to be treated, and it is possible to prepare a therapeutic agent that is specifically adapted/optimized to the appropriate indication. Overall, the selectivity of the TNF receptor ligand action with the present polypeptides according to the invention is thus achieved via two mechanisms: on the one hand via the concentration, mediated by the cell surface molecule binding domain, of the fusion protein that is inactive or of restricted activity in the unbound state, and, more importantly, via its activation that depends on the cell surface molecule binding, i.e. immobilization.

The present invention is illustrated in greater detail by the Figures below.

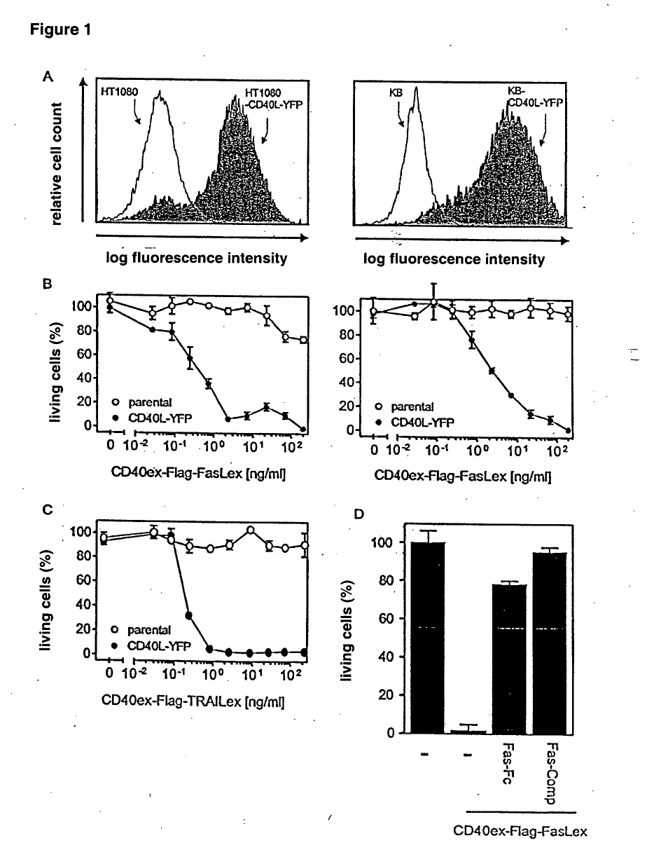

FIG. 1 shows the results of experiments on the preferential induction of apoptosis by fusion proteins according to the invention, using CD40L-positive cells as an example. To make the CD40L expression easily detectable, yellow fluorescent protein, YFP, was fused to the intracellular domain of CD40L.

FIG. 1A shows the expression of the CD40L-YFP fusion protein in parental KB and HT1080 cells and in the transfectands derived therefrom that stably express CD40L-YFP, by means of FACS analysis. A corresponding picture is also obtained by means of FACS analyses with antibodies that recognize the extracellular domain of CD40L (data not shown).

FIG. 1B to 1D are plots of the cell vitality (in %) against the concentration of the fusion protein indicated in each case. The curves drawn in represent the results of the treatment of CD40L-positive (HT1080-CD40L, KB-CD40L) or CD40L-negative HT1080 and KB cells with the constructs CD40ex-Flag-FasLex and CD40ex-Flag-TRAILex.

In the experiments according to FIG. 1B, HT1080 and HT1080-CD40-YFP cells (left-hand diagram) and KB and KB-CD40L-YFP cells (right-hand diagram) were cultivated overnight in a 96-well cell culture dish. The next day, the cells were stimulated with the indicated concentrations of CD40ex-Flag-FasLex. The cells were treated in parallel with CHX (2.5 μg/ml) for sensitization of the induction of apoptosis. The following day, the cell vitality was determined by means of crystal violet staining.

In the experiments according to FIG. 1C, HT1080 and HT1080-CD40L-YFP cells were cultivated overnight in a 96-well cell culture dish. The next day, the cells were stimulated with the indicated concentrations of CD40ex-Flag-TRAILex. The procedure was otherwise as described for FIG. 1B.

FIG. 1D shows by way of example that the cell death induced by CD40ex-Flag-FasLex in CD40L-expressing HT1080 cells is mediated by Fas. Once again, HT1080-CD40L-YFP cells were cultivated overnight in a 96-well cell culture dish. The next day, the cells were stimulated with 100 ng/ml of CD40ex-Flag-FasLex. Where indicated, the CD40ex-Flag-FasLex reagent was first preincubated with 2 μg/ml of Fas-Fc or Fas-Comp. The procedure was otherwise as described for FIG. 1B or 1C. By masking the FasL part in CD40ex-Flag-FasLex with the soluble FasL-binding reagents Fas-Fc and Fas-Comp, it was possible partially or completely to block the induction of apoptosis on CD40L-positive cells.

The present invention is illustrated in greater detail by means of the Examples which follow.

### EXAMPLE 1

Construction of the Polypeptides CD40ex-Flag-FasLex and CD40ex-Flag-TRAILex According to the Invention

Construct (A): CD40ex-Flag-FasLex

NH₂— [CD40(1-192)]-[linker with Flag-tag]-[FasL(139-281)]-COOH
CD40(1-192): extracellular domain of the human CD40 receptor, including leader sequence (AA 1-192)
Linker1: linker with Flag epitope (emboldened)
   GSDYKDDDDKEFGRGDSPGRGDSP
FasL(139-281): extracellular domain of human FasL (AA 139-281)
   Construct (B): CD40ex-Flag-TRAILex
NH₂-[CD40(1-192)]-[linker=Flag-tag]-[TRAIL(95-281)]-COOH
CD40(1-192): extracellular domain of the human CD40 receptor, including leader sequence (AA 1-192)
Linker1: linker with Flag epitope (emboldened)
   GSDYKDDDDKEF
TRAIL(95-281): extracellular domain of human
   TRAIL (AA 95-281)
      The fusion proteins were prepared as follows:
      CD40ex-Flag-FasLex:
1. The cDNA corresponding to the extracellular domain of the CD40 receptor (amino acids 1-192, incl. leader sequence) was amplified from a cDNA pool of activated B cells by means of proofreading PCR. With the aid of the primers used (no. 1160 and no. 1161), a Hind3 cleavage (restriction) site was inserted at the 5′ end of the amplicon and a BamH1 cleavage (restriction) site was inserted at the 3′ end. The amplicon digested with Hind3 and BamH1 was then introduced into eukaryotic expression vector pCR3 (Invitrogen), likewise digested with Hind3 and BamH1.
2. The cDNA corresponding to the extracellular domain of FasL (amino acids 139-281) was amplified from a cDNA pool of activated T cells by means of proofreading PCR. With the aid of the primers used (no. 1106 and no. 1107), an EcoR1 cleavage (restriction) site was inserted at the 5′ end of the amplicon and an Xho1 cleavage (restriction) site was inserted at the 3′ end. By means of the forward primer, six amino acids were also introduced as a linker. The amplicon digested with EcoR1 and Xho1 was then introduced into the intermediate obtained in 1., likewise digested with EcoR1 and Xho1.
3. Finally, for analytical purposes, a Flag-tag was inserted by means of oligonucleotide linkers between the BamH1 and EcoR1 cleavage (restriction) sites of the intermediate obtained in 2. The resulting construct (pCR3—CD40ex-Flag-FasLex) codes for a fusion protein with the extracellular domain of the CD40 molecule and the extracellular domain of the Fas ligand, which are bound by a Flag-tag and some other AA.
   CD40ex-Flag-TRAILex:
1. The cDNA corresponding to the extracellular domain of TRAIL (amino acids 95-281) was amplified from a cDNA pool of activated T cells by means of proofreading PCR. With the aid of the primers used (no. 1302 and no. 1334), an EcoR1 cleavage (restriction) site was inserted at the 5′ end of the amplicon and an Xba1 cleavage (restriction) site was inserted at the 3′ end. The amplicon digested with EcoR1 and Xba1 was then introduced into expression vector pCR3—CD40ex-Flag-FasL-ex, likewise digested with EcoR1 and Xba1. The FasL fragment freed from pCR3—CD40ex-Flag-FasL-ex by digestion was removed prior to ligation. The resulting construct (PCR3—CD40ex-Flag-TRAILex) codes for a fusion protein with the extracellular domain of the CD40 molecule and the extracellular domain of the TRAIL molecule, which are bound by a Flag-tag and some other AA.

To obtain CD40ex-Flag-FasLex and CD40ex-Flag-TRAILex, HEK293 or Cos7 cells with the above-described constructs were transfected with lipofectamine (Gibco-BRL) according to the manufacturer's instructions. 48-96 hours after transfection, the fusion protein supernatants were sterile-filtered and stored at 4° C. until required for further use.

All the cloning and PCR amplification steps were carried out by conventional standard procedures with the primers below. All the constructs were sequenced for verification of the cDNA sequence.

Primer 1106
5′CCG GAA TTC GGC CGG GGC GAC TCA CCC GGC CGG GGC
GAC TCA CCC GAA AAA AAG GAG CTG AGG AAA GTG GCC 3′
Primer 1107
5′CCG CTC GAG GTG CTT CTC TTA GAG CTT ATA TAA GCC
G 3′
Primer 1160
5′CCC AAG CTT CTC GCC ATG GTT CGT CTG CCT CTG CAG
3′
Primer 1161
5′CGC GGA TCC CAG CCG ATC CTG GGG ACC ACA GAC 3′
Primer 1302
5′CCG GAA TTC TAC GCA TAT TAC ACC TCT GAG GAA ACC
ATT TCT
ACA G 3′
Primer 1334
5′TGC TCT AGA CCA GGT CAG TTA GCC AAC TAA AAA GGC
3′

### Example 2

Detection of the CD40L-dependent activation of CD40ex-Flag-FasLex and CD40ex-Flag-TRAILex using CD40L-expressing cells as an example (cf. FIG. 1A)

CD40ex-Flag-FasLex and CD40ex-Flag-TRAILex were provided as described in Example 1. CD40L-expressing cells (HT1080-CD40L; KB-CD40L-YFP) and CD40L-negative cells (HT1080; KB) were cultivated overnight in 96-well cell culture dishes. The next day, the cells were incubated for 8 h with the indicated concentrations of CD40ex-Flag-FasLex and CD40ex-Flag-TRAILex in the presence of CHX (2.5 μg/ml). The surviving cells were quantified by means of crystal violet staining.

1. Polypeptide comprising a segment (1), which contains a cell surface molecule binding domain, a segment (2), which is a peptide linker, and a segment (3), which contains a fragment of a member of the TNF ligand family that as such is biologically inactive/of restricted activity, the cell surface molecule binding domain not being derived from an immunoglobulin and the fragment of the member of the TNF ligand family becoming biologically fully active only when segment (1) binds to the cell surface molecule.

2. Polypeptide according to claim 1 wherein segments (1) to (3) are arranged from N-terminal to C-terminal.

3. Polypeptide according to claim 1 wherein segment (3) contains the extracellular domain, a functional variant of the extracellular domain or a functional fragment of the extracellular domain of the member of the TNF ligand family.

4. Polypeptide according to claim 3 wherein segment (3) contains the extracellular domain of TRAIL, FasL, TNF, 41BBL, CD40L, CD30L or Ox40L or a functional variant or a functional fragment of said extracellular domains.

5. Polypeptide according to one claim 1 wherein the cell surface molecule binding domain binds to a membrane protein.

6. Polypeptide according to claim 5 wherein the cell surface molecule binding domain comprises at least that segment of a ligand of a membrane-based receptor which is required for the specific interaction with the cell surface molecule.

7. Polypeptide according to claim 6 wherein the ligand is a receptor-binding peptide or protein hormone or a receptor-binding fragment of a cytokine.

8. Polypeptide according to claim 7 wherein the cytokine fragment is a fragment of a member of the TNF ligand family, with the proviso that the member of the TNF ligand family from which segments (1) and (3) are derived is different.

9. Polypeptide according to claim 7 wherein the hormone is selected from the group consisting of growth factors, especially EGF, and angiogenesis factors, especially VEGF.

10. Polypeptide according to claim 5 wherein the cell surface molecule binding domain comprises at least that segment of a receptor for a membrane-based ligand which is required for the specific interaction with the cell surface molecule, with the proviso that the receptor segment does not bind to the extracellular domain of the member of the TNF ligand family present in segment (3).

11. Polypeptide according to claim 10 wherein the receptor is selected from the TNF receptor superfamily.

12. Polypeptide according to claim 10 wherein the receptor is selected from the group consisting of TNF-R2, CD30, CD40 and CD28.

13. Nucleic acid construct containing a nucleotide sequence that codes for a polypeptide according to claim 1.

14. Vector containing the nucleic acid construct according to claim 13.

15. Host cell containing a material selected from the group consisting of the nucleic acid construct according to claim 13, a vector containing said nucleic acid construct, and both said nucleic acid construct and said vector.

16. Process for the isolation of a polypeptide according to claim 1, wherein
(a) a nucleic acid construct containing a nucleotide sequence that codes for said polypeptide, or a vector containing said nucleic acid construct is prepared,
(b) cells are transfected with a vector or nucleic acid construct obtained according to process step (a),
(c) the cells transfected according to (b) are cultivated, and
(d) polypeptides expressed under appropriate conditions are isolated from the host cells and/or the culture supernatant.

17. A method for the treatment of cancer diseases, especially solid or lymphatic tumours, infectious diseases, metabolic diseases, inflammatory states and autoimmune diseases, especially rheumatic/arthritic diseases, comprising administration to a subject in need thereof, a material selected from the group consisting of a polypeptide according to claim 1; a nucleic acid construct containing a nucleotide sequence that codes for said polypeptide; a vector containing said nucleic acid construct; and a host cell, said host cell containing a material selected from the group consisting of said nucleic acid construct and said vector.

18. Pharmaceutical composition containing a material selected from the group consisting of a polypeptide according to claim 1; a nucleic acid construct containing a nucleotide sequence that codes for said polypeptide; a vector containing said nucleic acid construct; and a host cell, said host cell containing a material selected from the group consisting of said nucleic acid construct and said vector, together with pharmaceutically acceptable auxiliary substances, additives and/or excipients.

19. Pharmaceutical composition according to claim 18 for the treatment of cancer diseases, especially solid or lymphatic tumours, infectious diseases, metabolic diseases, inflammatory states and autoimmune diseases, especially rheumatic/arthritic diseases.
